(19) United States
(12) Patent Application Publication
(10) Pub. No.: US 2004/0157784 A1
(43) Pub. Date: Aug. 12, 2004

(54) Opiod tannate compositions

(75) Inventors: Vilas M. Chopdekar, Edison, NJ (US); Sham N. Redkar, Bound Brook, NJ (US); James R. Schlek, Somerset, NJ (US)

Correspondence Address: Jack Matalon, Attorney at Law, 32 Shelley Rd., Springfield, NJ, 07081-2529, US

(73) Assignee: Jame Fine Chemicals, Inc.

(21) Appl. No.: 10/734,460

(22) Filed: Dec. 12, 2003

Related U.S. Application Data
(60) Provisional application No. 60/446,230, filed on Feb. 10, 2003.

(51) Int. Cl. A61K 31/70; A61K 31/522; A61K 31/60; A61K 31/485; A61K 31/46

(52) U.S. Cl. 514/23; 514/165; 514/263.32; 514/282; 514/304

(57) ABSTRACT

A composition comprising the tannate of an opioid. Suitable opioids include alfentanil, buprenorphine, butorphanol, carfentanil, cocaine, codeine, dezocine, diacetylmorphine, dihydrocodeine, dihydromorphine, diphenoxylate, diprenorphine, etorphine, fentanyl, heroin, hydrocodone, hydromorphone, β-hydroxy-3-methylfentanyl, levo-α-acetylmethadol, levorphanol, lofentanil, meperidine, methadone, morphine, nalbuphine, nalmefene, o-methylnaltrexone, naloxone, naltrexone, oxycodone, oxymorphone, pentazocine, pethidine, propoxyphene, remifentanil, sufentanil, tilidine and tramadol. The opioid tannate may be readily prepared by reacting an opioid free base with tannic acid, either neat or in the presence of up to about 30 wt. % water, at a temperature of about 60 to about 150° C. and thereafter recovering the resultant opioid tannate. The opioid tannate may also be prepared by an alternative process that involves reacting the opioid free base with water at a temperature such that not more than about 10 wt. % of the opioid tannate will be decomposed and thereafter removing the water by freeze-drying.

### RELATED APPLICATIONS

This application is a continuation-in-part of provisional application Serial No. 60/446,230 filed Feb. 10, 2003.

### FIELD OF THE INVENTION

The invention relates to compositions comprising opioid tannates and to methods for preparing such opioid tannates.

### BACKGROUND OF THE INVENTION

The term “opioid” is understood to refer to naturally occurring and synthetic analgesics (i.e., pain-relievers) with chemical structures and actions similar to morphine. Opioids are frequently referred to as narcotics and are commonly prescribed because of their effective analgesic or pain-relieving properties. Of course, opioids are classified as “controlled substances” by federal and state drug enforcement agencies and care must be taken to insure that they are prescribed for the particular pain suffered by the patient and not indiscriminately used to obtain a “high.”

Opioids act by attaching to specific proteins termed opioid receptors that are found in the brain, spinal cord and gastrointestinal tract. Opioids interfere with, and stop the transmission of, pain messages to the brain. Opioids do not take the pain away, but they do reduce and alter the patient's perception of the pain.

It is also thought that opioids mimic the effects of morphine-like substances in the body termed endorphins. Endorphins are produced at various sites in the body and function presumably as the body's natural defense against pain. Endorphins act by attaching themselves at specific sites on the outside of neurons referred to as opioid receptors. After they occupy the receptors, they stimulate a chain of reactions that results in a depression of their normal activity for a short time. They then leave the receptors and the normal functions of the neurons return.

Opioids, i.e., opioid drugs, have the same depressant effects as endorphins because they look similar and are able to fit within the opioid receptor without it realizing that it is not endorphin. When this occurs on a nerve that is responsible for transmitting information about pain to the brain from the site where the painful event is coming from, the effect will be pain relief. When it occurs on other neurons, then the effect will be to depress whatever the normal function of that neuron is.

There are several major concerns associated with the use of opioids. The first relates to the dose—the higher the dose given, the stronger the effect. This holds for both pain relief and side effects. The principal side effects are constipation and respiratory depression. While the side effects can be somewhat ameliorated by reducing the dosage, the desired analgesic effect is also proportionately reduced. It would be most desirable if the opioid could be prescribed in a dosage form that provides for the desired level of analgesic effect over an extended period of time without producing “bursts” of pain relief.

A second major concern with the use of opioids is that the patient may become addicted to the prescribed opioid. The likelihood of addiction is increased if multiple doses of the opioid must be taken over a period of time in order to achieve continuous pain relief. Here again, it would be most desirable if the prescribed opioid had extended release properties thereby providing pain relief over an extended period of time, thus eliminating or reducing the frequency of multiple doses and concurrently reducing the likelihood of addition.

Opioid drugs currently available do not have extended release properties. Such currently available opioids are typically available in the form of salts (in order to provide for aqueous solubility) such as hydrochlorides, hydrobromides, hydroiodides, sulfates, citrates, maleates, etc. These salts are readily converted into the free base forms of the opioids in the body thereby rapidly providing the desired analgesic effect. However, the analgesic effect is short-lived and multiple doses are required to maintain the analgesic regimen at the desired level.

A third major concern associated with opioids is that they are prone to abuse. An opioid such as oxycodone hydrochloride is frequently abused by crushing the tablet and inhaling the resultant powder through the nasal passages. Alternatively, oxycodone hydrochloride may be dissolved it in water at moderate temperatures (e.g., by heating the crushed tablet with a small amount of water in a spoon held over a candle flame). The resultant solution may then be injected. The opioid tannates of the present invention overcome such type of abuse. When the opioid tannates of the invention are mixed with water, a gummy paste results. Such paste cannot be inhaled since the paste would block the nasal passages. Furthermore, the viscosity of the paste is such that it will not flow through a hypodermic needle. Moreover, attempts to extract the opioid base from the opioid tannate composition have not been successful to date.

### OBJECTS OF THE INVENTION

It is an object of the invention to provide opioid compositions that have extended-release properties.

It is also an object of the invention to provide opioid compositions that are not prone to drug abuse.

It is an additional object of the invention to provide a “hot-melt” method for preparing opioid compositions that have extended-release properties.

It is a further object of the invention to provide a “freeze-dry” method for preparing opioid compositions that have extended release properties.

The foregoing objects will be met by providing opioid tannates prepared in accordance with the details as set forth below.

### DETAILED DESCRIPTION OF THE INVENTION

In its broadest aspect, the invention is directed to a composition comprising the tannate of an opioid and to processes for preparing such compositions. For the purposes of the present invention, the opioid may be any of those that are readily commercially available such as alfentanil, buprenorphine, butorphanol, carfentanil, cocaine, codeine, dezocine, diacetylmorphine, dihydrocodeine, dihydromorphine, diphenoxylate, diprenorphine, etorphine, fentanyl, heroin, hydrocodone, hydromorphone, β-hydroxy-3-methylfentanyl, levo-α-acetylmethadol, levorphanol, lofentanil, meperidine, methadone, morphine, nalbuphine, nalmefene, o-methylnaltrexone, naloxone, naltrexone, oxycodone, oxymorphone, pentazocine, pethidine, propoxyphene, remifentanil, sufentanil, tilidine and tramadol. All of these opioids are well known in the prior art and descriptions of their physical and pharmacological properties may be found in reference texts such as Physician's Desk Reference and The Merck Index. Further information regarding these opioids may be found using an internet search engine and searching under the topic “Opioids.”

Preferably the opioid is one or more of the following: codeine, diacetylmorphine, dihydrocodeine, hydrocodone, hydromorphone, meperidine, methadone, morphine, oxycodone, oxymorphone and propoxyphene.

The opioid tannate compositions of the invention may be administered in the form of a tablet, a gel, a liquid suspension, a suppository, etc. Tablets containing the unique opioid tannate of the invention may be prepared in a conventional manner by the addition of suitable pharmaceutical carriers, including fillers, diluents, lubricants and the like as well as conventional and well-known binding and disintegrating agents. A typical tablet composition of the present invention will contain, in addition to the opioid tannate, microcrystalline cellulose, corn starch, magnesium stearate, croscarmellose sodium and coloring matter.

The suspension formulations of the opioid tannate of the present invention will typically additionally contain citric acid, caramel, glycerin, sorbitol solution, propylene glycol, saccharin sodium, sodium benzoate, flavoring agent and purified water.

If desired, the opioid tannate composition of the invention may be formulated with other pharmaceutically active ingredients such as expectorants, decongestants, antihistamines and antitussives, e.g., dextromethorphan, diphenhydramine, chlorpheniramine, brompheniramine, dexchlorpheniramine, dextrobrompheniramine, pyrilamine, phenylephrine, ephedrine, pseudoephedrine, carbetapentane, carbinoxamine, guaifenesin, and the like. Typically, these other active ingredients may be employed in the form of their free bases or as their salts, e.g., citrates, maleates, hydrobromides, hydrochlorides, tannates, etc. Of course, the dosage of the opioid tannate of the present invention, alone or in combination with other pharmaceutically active ingredients to be administered, will be dependent on the age, health and weight of the recipient, types of concurrent treatment, if any, frequency of treatment and effect desired.

The opioid tannates of the invention may be readily prepared by either the “hot-melt” process or the “freeze-dry” process described below. Both processes start with the opioid being present in the form of its free base. If the opioid is present in the form or a salt such as a hydrochloride, sulfate, maleate, citrate, etc., the opioid must first be converted to its free base form. This is readily accomplished by treating the opioid salt with an aqueous solution of a base such as sodium hydroxide in an amount to liberate the opioid free base. The opioid free base is then washed free of the sodium salt that is the byproduct of the neutralization reaction.

In the hot-melt process, the opioid free base is reacted with tannic acid at a temperature of about 60 to about 150° C., preferably 70 to 130° C., and thereafter the resultant opioid tannate is recovered. The reaction time is typically in the range of several minutes to four hours. Although the reaction may be carried out “neat,” the reaction mass may become too viscous to stir, even though commercially available tannic acid contains about 5 wt. % water. Accordingly, water in the amount of up to about 30 wt. % may be added to facilitate stirring. Preferably, only small amounts of additional water, e.g., 5 wt. %, are added since smaller amounts of additional water facilitate drying of the opioid tannate product. Typically, the opioid free base is employed in an amount of about 3 to about 8 moles, preferably 5 to 6, moles of the free base per mole of tannic acid.

The resultant opioid tannate is then recovered and dried to the desired extent by conventional methods, e.g., vacuum oven at 50-75° C. for several hours, heat lamps, sparging with nitrogen, etc. The dried opioid tannate may then be milled to a free-flowing powder, typically to a particle size of about 50 to 200 mesh.

The “freeze-dry” process for preparing the opioid tannates of the invention involves the following steps:

(1) contacting an opioid in the form of its free base with tannic acid in the presence of water at a maximum temperature that will not cause decomposition of the opioid tannate to an extent of greater than about 10 wt. %, based on the weight of the opioid tannate;

(2) allowing the opioid to remain in contact with the tannic acid in the presence of water for a period of time ranging from about 5 minutes to about 24 hours at said maximum temperature; and

(3) freeze-drying the opioid tannate resulting from step (2) at a temperature and at a reduced pressure such that (i) at least about 80 wt. % of the water is removed from the opioid tannate and (ii) decomposition of the opioid tannate will be limited to a maximum of about 10 wt. %, based on the weight of the opioid tannate.

In the freeze-dry process, the opioid free base is typically employed in an amount of about 3 to about 8 moles, preferably 5 to 6 moles, of the free base per mole of tannic acid. In general, the amount of water is present in an amount such that the weight ratio of tannic acid to water is in the range of about 1:10 to about 10:1. The contact time in step (2) is generally in the range of about 15 minutes to about 4 hours. Steps (1) and (2) are generally carried out at a temperature in the range of about 20 to about 85° C., preferably 20 to 50° C.

The freeze-drying step, i.e., step (3), is generally carried out at a pressure of not greater than about 500 milliTorre and at a temperature in the range of about −60° C. to about −20° C., preferably at a pressure of 300 to 100 milliTorre and at a temperature of −50° C. to −40° C. The resultant opioid tannate from step (3) may be milled to provide a free-flowing powder, such that the powder will have a particle size in the range of about 50 to about 200 mesh.

If decomposition of the opioid tannate is avoided (or at least minimized) in the course of its preparation, the principal “impurity” in the opioid tannates of the invention will be water, regardless of whether the method employed to prepare the opioid tannates is the hot-melt or the freeze-dry method. Accordingly, the amount of moisture remaining in the opioid tannates of the invention after completing the method of preparation is somewhat irrelevant, and will depend upon the selected method of administration of the opioid tannate of choice. If the opioid tannate of choice is to be orally administered in the form of a tablet, the opioid tannate would be dried to a relatively low moisture content, e.g., 5 wt. % or lower. Suspensions and suppositories will generally entail the use of an opioid tannate with a moisture level of about 10-15 wt. %. In any event, regardless of the selected form of administration, the moisture content of the opioid tannate should be taken into account when the dose is formulated.

The following nonlimiting examples shall serve to illustrate the various embodiments of this invention. Unless otherwise stated to the contrary, all parts and percentages are on a weight basis.

### EXAMPLE 1

### Preparation of Hydrocodone Free Base

A five-liter round bottom flask was fitted with a stirrer, thermometer, dropping funnel and water bath. 412.6 g (0.83 mole) of hydrocodone bitartrate and 3.3 kg of purified water were added to the flask and the mixture was stirred at a temperature of 30-40° C. To the resultant solution were added 310 g of a 20% aqueous solution of sodium hydroxide through the dropping funnel over a period of about one hour, while stirring and maintaining a temperature of 30-40° C. At this point, the pH of the reaction mixture measured 12-13. The reaction mixture was allowed to settle and the supernatant liquid was decanted off. About 2 liters of purified water were added to the solid in the flask and the mixture was stirred for 15 minutes. The solid was filtered off and washed with two liter portions of purified water. The solid was sucked dry and it weighed 290.4 g. A small sample of the solid was dried under a heat lamp and it was determined that the melting point of the hydrocodone free base was 198-199° C.

### EXAMPLE 2

### Synthesis of Hydrocodone Tannate by the Hot-Melt Process

A 300 ml beaker was set up with a magnetic stirrer, thermometer, oil bath and a hot plate. The oil bath was heated to a temperature of 100-110° C. and 8 g of purified water and 34 g (0.02 mole) of tannic acid having a K.F. moisture content of 4.8% were charged to the beaker, while stirring. Thereafter, 35.4 g (29.94 g on a dry basis equivalent to 0.1 mole) of hydrocodone free base having a K.F. moisture content of 15.5% prepared in Example 1 were added to the beaker over a period of about 30 minutes, while stirring and maintaining a temperature of 100-110° C. The reaction mixture was stirred at this temperature for an additional 30 minutes and then poured into a dish and allowed to cool over a period of 2 hours. The solid was pulverized and it weighed 70.9 g.

The solid was determined to have a K.F. moisture content of 9.2%. After the solid was dried in a vacuum oven at 60-70° C. for three hours, the K.F. moisture content was determined to be 1.1%. A reaction completion test was then carried out on an aliquot sample of the product as follows, using methylene chloride as the solvent since the hydrocodone free base is soluble to the extent of 99.94%. A sample of 2.107 g of the dried reaction product was mixed with 100 ml of methylene chloride in a small beaker for 10 minutes. The reaction mixture was gravity filtered into a 250 ml round bottom flask. The beaker was rinsed with three 20 ml portions of methylene chloride and poured through the filter paper into the flask. The filtrate in the flask was evaporated to dryness over a period of 30-45 minutes at 35-40° C., purged with nitrogen and allowed to stand for 5-10 minutes. The residue in the flask weighed 0.0104 g corresponding to 0.49% hydrocodone free base. Thus, the reaction completion for the hydrocodone tannate made by the hot melt process was determined to be 99.51%.

### EXAMPLE 3

### Synthesis of Hydrocodone Tannate by the Freeze-Dry Process

A 500 ml beaker was set up with a magnetic stirrer, thermometer and water bath. The water bath was heated to a temperature of about 65° C. and 70 g of purified water and 100.8 g (0.056 mole) of tannic acid (K.F. moisture level of 4.8%) were charged to the beaker and the contents were stirred for a few minutes. Thereafter, 100 g (84.5 g on a dry basis equivalent to 0.28 mole) of hydrocodone free base having a K.F. moisture content of 15.5% prepared in Example 1 were added to the beaker over a period of about 30 minutes, while stirring and maintaining a temperature of about 65° C. The reaction mixture was stirred for one additional hour and the viscous reaction mixture was poured onto a stainless steel tray and freeze-dried at a pressure of about 500 milliTorre and a temperature of about −50° C. The yield of the product was 190.72 g. The percentage of reaction completion was determined to be 99.73% by the methylene chloride solvent method described in Example 2.

The softening points of hydrocodone tannate prepared in Examples 2 and 3 were as follows:

K.F. moisture content of 9.2%: 68-78° C.

K.F. moisture content of 4.9%: 77-87° C.

K.F. moisture content of 1.1%: 95-105° C.

### EXAMPLE 4

### Synthesis of Oxycodone Tannate by the Hot-Melt Process

Example 2 is repeated using 12 g of purified water, 38 g (0.02 mole) of tannic acid (K.F. moisture content of 4.8%) and 31.5 g (0.1 mole) of oxycodone base obtained from a commercial source such as Halsey Pharmaceutical Co. of Congers, N.Y. The yield of the oxycodone tannate will be 71 g and the reaction completion percentage will be comparable to that of the hydrocodone tannate prepared in Example 2.

### EXAMPLE 5

### Synthesis of Oxycodone by the Freeze-Dry Process

Example 3 is repeated using 80 g of purified water, 114 g (0.06 mole) of tannic acid (K.F. moisture content of 4.8% and 94.5 g (0.3 mole) of oxycodone base (K.F. moisture content of 5%) obtained from a commercial source such as Halsey Pharmaceutical Co. of Congers, N.Y. The yield of the oxycodone tannate will be 210 g and the reaction completion percentage will be comparable to that of the hydrocodone tannate prepared in Example 3.

What is claimed is:

1. A composition comprising the tannate of an opioid.

2. The composition of claim 1 wherein the opioid is selected from the group consisting of alfentanil, buprenorphine, butorphanol, carfentanil, cocaine, codeine, dezocine, diacetylmorphine, dihydrocodeine, dihydromorphine, diphenoxylate, diprenorphine, etorphine, fentanyl, heroin, hydrocodone, hydromorphone, β-hydroxy-3-methylfentantanyl, levo-α-acetylmethadol, levorphanol, lofentanil, meperidine, methadone, morphine, nalbuphine, nalmefene, o-methylnaltrexone, naloxone, naltrexone, oxycodone, oxymorphone, pentazocine, pethidine, propoxyphene, remifentanil, sufentanil, tilidine and tramadol.

3. The composition of claim 2 wherein the opioid is selected from the group consisting of codeine, diacetylmorphine, dihydrocodeine, hydrocodone, hydromorphone, meperidine, methadone, morphine, oxycodone, oxymorphone and propoxyphene.

4. The composition of claim 3 wherein the opioid comprises hydrocodone.

5. The composition of claim 3 wherein the opioid comprises oxycodone.

6. The composition of claim 3 further comprising acetaminophen.

7. The composition of claim 7 further comprising aspirin and caffeine.

8. An analgesic composition comprising a pharmaceutically effective amount of an ingredient comprising an opioid as claimed in claim 1

9. The analgesic composition of claim 8 in the form of an injectable solution, a tablet, a gel, a liquid suspension or a suppository.

10. The analgesic composition as claimed in claim 8 further comprising one or more expectorant, decongestant, antihistaminic, antitussive, and/or non-steroidal anti-inflammatory compositions.

11. A method for relieving pain in a human being that comprises administering to such human being a pharmaceutically effective amount of a composition as claimed in claim 1.

12. A method for preparing an opioid tannate that comprises reacting an opioid free base with tannic acid at a temperature of about 60 to about 150° C. and thereafter recovering the resultant opioid tannate.

13. The method of claim 12 wherein the opioid free base is employed in an amount of about 4 to about 8 moles of the freebase per mole of tannic acid.

14. The method of claim 12 wherein the reaction is carried out in the presence of up to about 30 wt. % water.

15. The method of claim 12 wherein the resultant opioid tannate is milled to provide a free-flowing powder having a particle size in the range of about 50 to about 200 mesh.

16. A method for preparing an opioid tannate comprising the steps of:
(1) contacting an opioid in the form of its free base with tannic acid in the presence of water at a maximum temperature that will not cause decomposition of the opioid tannate to an extent of greater than about 10 wt. %, based on the weight of the opioid tannate;
(2) allowing the opioid to remain in contact with the tannic acid in the presence of water for a period of time ranging from about 5 minutes to about 24 hours at said maximum temperature; and
(3) freeze-drying the opioid tannate resulting from step (2) at a temperature and at a reduced pressure such that (i) at least about 80 wt. % of the water is removed from the opioid tannate and (ii) decomposition of the opioid tannate will be limited to a maximum of about 10 wt. %, based on the weight of the opioid tannate.

17. The method of claim 16 wherein the opioid free base is employed in an amount of about 4 to about 8 moles of the free base per mole of tannic acid.

18. The method of claim 16 wherein steps (1) and (2) are carried out at a temperature in the range of about 20 to about 85° C.

19. The method of claim 16 wherein step (3) is carried out at a pressure of not greater than about 500 milliTorre and at a temperature in the range of about −60° C. to about −20° C.

20. The method of claim 16 wherein the opioid tannate from step (3) is milled to provide a free-flowing powder having a particle size in the range of about 50 to about 200 mesh.
